(19) United States
(12) Patent Application Publication
(10) Pub. No.: US 2024/0016726 A1
(43) Pub. Date: Jan. 18, 2024

(54) OIL-IN-WATER EMULSION COSMETIC

(71) Applicant: Shiseido Company, Ltd., Chuo-ku, Tokyo (JP)

(72) Inventors: Keita NISHIDA, Tokyo (JP); Kento UENO, Tokyo (JP)

(73) Assignee: Shiseido Company, Ltd., Chuo-ku, Tokyo (JP)

(21) Appl. No.: 18/038,892

(22) PCT Filed: Aug. 16, 2021
(86) PCT No.: PCT/JP2021/029917

§ 371 Date: May 25, 2023

(30) Foreign Application Priority Data
Dec. 25, 2020 (WO) PCT/JP2020/048754

(51) Int. Cl. A61K 8/92; A61K 8/06

(52) U.S. Cl. CPC A61K 8/92; A61K 8/062; A61K 2800/5424; A61K 2800/43

(57) ABSTRACT

An objective of the present invention is to provide an oil-in-water emulsion cosmetic in which a UV absorbing agent and a pigment can be stably blended, having excellent vibration stability while also having improved makeup effects such as compatibility with skin, uniformity of the coating film, and non-thickly-coated appearance. The oil-in-water emulsion cosmetic of the present invention contains (A) a vesicle-forming amphiphilic substance, (B) an oil component containing a UV absorbing agent other than ethylhexyl methoxycinnamate, and (C) an amino acid surface-treated pigment.

### TECHNICAL FIELD

The present invention relates to an oil-in-water emulsion cosmetic in which a UV absorbing agent and a pigment can be stably blended into an emulsion containing a vesicle-forming amphiphilic substance as an emulsifier, and which has improved makeup effects, such as compatibility with skin, uniformity of the coating film, and non-thickly-coated appearance.

### BACKGROUND ART

Among amphiphilic substances, there are those that form spherical enclosed bodies comprising bimolecular films (lamellar liquid crystal) in an aqueous phase, and such bimolecular film-enclosed bodies are known as vesicles. Vesicles can hold water-soluble components inside the enclosed bodies and can hold oil-based components inside the bimolecular film. Since they can also be expected to provide effects such as improved system stability, they are used as bases in cosmetics.

For example, in Patent Document 1, an oil-in-water emulsion cosmetic containing a hydrated oil component in the internal phase is obtained by forming vesicles by means of polyoxyethylene hydrogenated castor oil, and adhering the vesicles to the surfaces of oil droplets. However, in the cosmetic in Patent Document 1, the problem of blending large amounts of pigments into the oil phase is not recognized, and it is unclear whether or not large amounts of pigments can be stably blended.

Meanwhile, in recent years, the use of ethylhexyl methoxycinnamate has become restricted in some regions of the world due to concerns over the effects thereof on the environment. Related to this fact, there is a demand, among some consumers, for cosmetics that do not contain ethylhexyl methoxycinnamate as a UV absorbing agent.

However, in the process of research by the present inventors, it has become clear that, if a base not containing ethylhexyl methoxycinnamate is used in a vesicle-containing oil-in-water emulsion cosmetic in which an inorganic powder is blended into the oil phase, which is the internal phase, then the vibration stability becomes poor.

Therefore, oil-in-water emulsion bases in which vesicle-forming amphiphilic substances are blended as emulsifiers have the problem of obtaining a cosmetic having excellent vibration stability while simultaneously obtaining high UV protection effects by blending a UV absorbing agent other than ethylhexyl methoxycinnamate and an inorganic powder such as a pigment into the oil phase.

### RELATED ART

### Patent Documents

Patent Document 1: JP 2011-195509 A

### SUMMARY OF THE INVENTION

### Problem to be Solved by the Invention

An objective of the present invention is to provide an oil-in-water emulsion cosmetic having excellent vibration stability, in which a UV absorbing agent other than ethylhexyl methoxycinnamate and a pigment can be stably blended into the oil phase.

### Means for Solving the Problem

The present inventors performed diligent research towards solving the aforementioned problem, as a result of which they discovered that, in an emulsion obtained by blending a vesicle-forming amphiphilic substance as the emulsifier, by subjecting a pigment blended into the oil phase to a surface hydrophobization treatment by an amino acid treatment, a base with excellent vibration stability can be obtained without including ethylhexyl methoxycinnamate, and simultaneously, a cosmetic with improved makeup effects, such as compatibility with skin, uniformity of the coating film, and non-thickly-coated appearance, can be obtained, thereby completing the present invention.

That is, the present invention provides an oil-in-water emulsion cosmetic containing:
(A) a vesicle-forming amphiphilic substance;
(B) an oil component containing a UV absorbing agent other than ethylhexyl methoxycinnamate; and
(C) an amino acid surface-treated pigment.

### Effects of the Invention

The cosmetic of the present invention, due to the above-mentioned features, can provide an emulsion containing a vesicle-forming amphiphilic substance as an emulsifier with excellent vibration stability even when not containing ethylhexyl methoxycinnamate, and can improve the makeup effects, such as compatibility with skin, uniformity of the coating film, and non-thickly-coated appearance. Thus, a cosmetic that is more suitable for being carried can be realized. Furthermore, since the emulsion base in the present invention has plenty of emulsifying power, it is possible to blend water-soluble agents, such as salt-type whiteners, that tend to have poor stability when blended into a water phase, which is usually the external phase.

### MODES FOR CARRYING OUT THE INVENTION

The cosmetic according to the present invention is characterized by containing (A) a vesicle-forming amphiphilic substance, (B) an oil component containing a UV absorbing agent other than ethylhexyl methoxycinnamate, and (C) an amino acid surface-treated pigment. The respective components constituting the cosmetic of the present invention will be explained in detail below.

### <(A) Vesicle-Forming Amphiphilic Substance>

The (A) vesicle-forming amphiphilic substance (hereinafter sometimes referred to simply as “component (A)”) blended into the cosmetic according to the present invention refers to an amphiphilic substance that forms vesicles, and that has emulsifying power. Though the vesicle-forming amphiphilic substances are not particularly limited, they include, for example, silicone-based surfactants, block-type alkylene oxide derivatives, sugar fatty acid esters, polyoxyethylene hydrogenated castor oil derivatives, acyl amino acid metal salts, phospholipids, etc.

### (Silicone-Based Surfactants)

Though the silicone-based surfactants are not particularly limited, they include, for example, polyoxyalkylene-modified silicones, etc. From the aspect of the texture in use and the vesicle-forming properties, a polyoxyalkylene-modified silicone indicated by Formula (1) below should preferably be used.

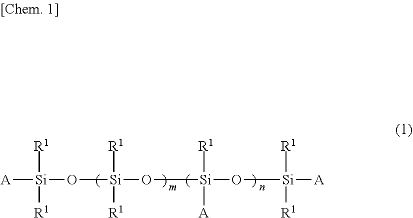

In Formula (1) above, the “R¹” units are side chains on the main-chain polysiloxane structures, and they may be hydrogen atoms or alkyl groups having 1 to 6 carbon atoms, which may be the same or individually different. For example, if the “R¹” units are all methyl groups, then a dimethyl polysiloxane structure is obtained, and if they are methyl groups and phenyl groups, then a methyl phenyl polysiloxane structure is obtained. The “A” units are sites where polyoxyalkylene groups can be introduced on the main-chain polysiloxane structure, at least one of which is a polyoxyalkylene group represented by —(CH₂)ₐ—(C₂H₄O)_{b}—(C₃H₆O)_{c}—R². In the formula, the “R²” unit is a hydrogen atom or an alkyl group having 1 to 6 carbon atoms, the subscript “a” represents a number from 1 to 6, the subscript “b” represents a number from 0 to 50, the subscript “c” represents an integer from 0 to 50, and b+c is at least 5 or greater.

In Formula (1) above, if some of the “A” units represent the above-mentioned polyoxyalkylene group, then the other “A” units may represent a hydrogen atom or an alkyl group having 1 to 6 carbon atoms. Regarding the silicone indicated in Formula (1), for example, if the two “A” units at the ends represent polyoxyalkylene groups, then the silicone becomes a polyoxyalkylene-modified silicone represented by the form A-B-A. On the other hand, if only the “A” units not at the ends represent polyoxyalkylene groups, then the silicone becomes a pendant-type polyoxyalkylene-modified silicone. The polyoxyalkylene group may be one of a polyoxyethylene group, a polyoxypropylene group, or a polyoxyethylene/polyoxypropylene group. The subscript “m”, which indicates the number of moles of unsubstituted polysiloxane structures, is an integer from 1 to 200, and the subscript “n”, which indicates the number of moles of polyoxyalkylene-substituted polysiloxane structures, is an integer from 0 to 50. If the subscript “n” represents 0, then one or both of the two “A” units at the ends must be a polyoxyalkylene group.

As such polyoxyalkylene-modified silicones, it is preferable to use, for example, polyoxyethylene(12 moles)-modified dimethyl polysiloxane, which is a pendant-type polyoxyalkylene-modified silicone having side-chain methyl groups on a linear dimethyl polysiloxane substituted by polyoxyethylene(12 moles) groups, or polyoxyethylene(8 moles)-modified dimethyl polysiloxane or polyoxyethylene(20 moles)-modified dimethyl polysiloxane. Aside from the above, examples include A-B-A type polyoxyethylene-methyl siloxane-polyoxyethylene block copolymers, etc. In the case in which a polyoxyethylene-modified silicone is used, the molecular weight occupied by the ethylene oxides within the overall molecular weight is preferably 20% to 60%.

The polyoxyalkylene-modified silicone in the present invention preferably has an HLB of less than 10 when calculating the HLB by Griffin's equation.

Among polyoxyalkylene-modified silicones, PEG-12 dimethicones in which the subscript “c” is 0 and the subscript “b” is 12 in the above formula are particularly preferred. Additionally, it is more preferable for the PEG-12 dimethicone to have an HLB of less than 10.

Examples of commercially available PEG-12 dimethicones include DOWSIL™ ES-5373; SH3772M, which has an HLB of 6, SH3773M, which has an HLB of 8, and SH3775M, which has an HLB of 5 (all manufactured by Dow Toray Co., Ltd.); and IM-22 (manufactured by Wacker Chemical Corp.).

### (Block-Type Alkylene Oxide Derivatives)

As block-type alkylene oxide derivatives, the block-type alkylene oxide derivatives indicated by Formula (2) or Formula (3) below may be used. The vesicles constituted by the block-type alkylene oxide derivatives are also called polymersomes.

R³O—[(EO)ₑ-(AO)_{f}-(EO)_{g}]—R⁴ (2)

In Formula (2) above, the “EO” units are oxyethylene groups and the “AO” units are oxyalkylene groups having 3 or 4 carbon atoms, these being added in the form of blocks. Specific examples of the “AO” units include oxypropylene groups, oxybutylene groups, oxyisobutylene groups, oxytrimethylene groups, oxytetramethylene groups, etc., among which oxypropylene groups and oxybutylene groups are preferred, and oxybutylene groups are particularly preferred.

In Formula (2) above, the subscripts “e” and “g” represent the average numbers of moles of oxyethylene groups that are added, and the subscript “f” represents the average number of moles of the oxyalkylene groups that are added. From the aspect of the stability of the vesicles, the texture in use, etc., “e” and “g” are preferably within the range 1≤e+g≤70 and more preferably within the range 5≤e+g≤60, and “f” is preferably within the range 1≤f≤70 and more preferably within the range 5≤f≤55.

In Formula (2) above, the percentage of the oxyethylene groups with respect to the total amount of the oxyalkylene groups having 3 or 4 carbon atoms and the oxyethylene groups is preferably 20% to 80% by mass and more preferably 30% to 70% by mass from the aspect of the vesicle-forming properties, etc.

The molecular weight of the alkylene oxide derivative represented by Formula (2) above is preferably 1000 to 5000 from the aspect of obtaining a sufficient amount of vesicles.

In Formula (2) above, the “R³” and “R⁴” units are hydrocarbon groups having 1 to 4 carbon atoms, and they may be the same or different. Examples of hydrocarbon groups having 1 to 4 carbon atoms include methyl groups, ethyl groups, N-propyl groups, isopropyl groups, N-butyl groups, sec-butyl groups, tert-butyl groups, etc., among which methyl groups and ethyl groups are preferable. The vesicles may be formed by one type or by a combination of two or more types of block-type alkylene oxide derivatives in which “R³” and “R⁴” are the same or different.

Within a range not causing problems in connection with the effects of the present invention, there may be derivatives in which one or both of “R³” and “R⁴” in the alkylene oxide derivative represented by Formula (2) is a hydrogen atom.

The block-type alkylene oxide derivative in the present invention can be manufactured by a known method. For example, the block-type alkylene oxide derivative can be obtained by addition-polymerizing ethylene oxides and alkylene oxides having 3 or 4 carbon atoms onto compounds having hydroxyl groups, then having the alkyl halides undergo an ether reaction in the presence of an alkaline catalyst.

Though the block-type alkylene oxide derivatives represented by Formula (2) above are not particularly limited, they include, for example, POE(9) POP(2) dimethyl ether, POE(14) POP(7) dimethyl ether, POE(10) POP(10) dimethyl ether, POE(6) POP(14) dimethyl ether, POE(15) POP(5) dimethyl ether, POE(25) POP(25) dimethyl ether, POE(7) POP(12) dimethyl ether, POE(22) POP(40) dimethyl ether, POE(35) POP(40) dimethyl ether, POE(50) POP(40) dimethyl ether, POE(55) POP(30) dimethyl ether, POE(30) POP(34) dimethyl ether, POE(25) POP(30) dimethyl ether, POE(27) POP(14) dimethyl ether, POE(55) POP(28) dimethyl ether, POE(36) POP(41) dimethyl ether, POE(17) POP(4) dimethyl ether, POE(9) POB(2) dimethyl ether, POE(14) POB(7) dimethyl ether, POE(15) POB(14) dimethyl ether, POE(18) POB(17) dimethyl ether, POE(23) POB(21) dimethyl ether, POE(27) POB(25) dimethyl ether, POE(32) POB(29) dimethyl ether, POE(35) POB(32) dimethyl ether, POE(10) POB(15) dimethyl ether, POE(20) POB(28) dimethyl ether, POE(17) POB(10) dimethyl ether, POE(28) POB(17) dimethyl ether, POE(45) POB(27) dimethyl ether, POE(34) POB(14) dimethyl ether, POE(55) POB(22) dimethyl ether, POE(44) POB(12) dimethyl ether, POE(10) POP(10) diethyl ether, POE(10) POP(10) dipropyl ether, POE(10) POP(10) dibutyl ether, POE(35) POP(30) glycol, POE(35) POB(32) glycol, etc. In this case, POE, POP and POB are respectively abbreviations for polyoxyethylene, polyoxypropylene and polyoxybutylene, and the numbers in parentheses after POE, POP and POB represent the respective number of moles added. Hereinafter, such abbreviations may be used.

R³O—[(EO)s¹-(AO)r¹]—B—[O(AO)r²-(EO)s²]—R⁴ (3)

In Formula (3), the “B” units are residues obtained by removing a hydroxyl group from a dimer diol and the “EO” units are oxyethylene groups. The “AO” units are oxyalkylene groups having 3 or 4 carbon atoms, including, for example, oxypropylene groups, oxybutylene groups, oxyisobutylene groups, oxy-t-butylene groups, etc., among which oxypropylene groups and oxybutylene groups are preferred and oxybutylene groups are more preferred. The “B”, “EO” and “AO” units may be added in the form of blocks from the aspect of the vesicle-forming properties. Regarding the sequence of addition, they should be bonded to a dimer diol in the order of “AO” followed by “EO”.

In Formula (3) above, “s¹” and “s²” represent the average number of moles of oxyethylene groups added, and “r¹” and “r²” represent the average number of moles of oxyalkylene groups added. From the aspect of the stability of the vesicles, the texture in use, etc., these should be within ranges such that 1≤s¹+s²≤150 and 1≤r¹+r²≤150, more preferably within ranges such that 5≤s¹+s²≤120 and 2≤r¹+r²≤70, and particularly preferably within ranges such that 10≤s¹+s²≤100 and 2≤r¹+r²≤50.

In Formula (3) above, the percentage of the oxyethylene groups with respect to the total amount of the oxyalkylene groups and the oxyethylene groups is preferably 10% to 99% by mass and more preferably 20% to 70% by mass from the aspect of the vesicle-forming properties, etc.

The molecular weight of the alkylene oxide derivative represented by Formula (3) above is preferably 1000 to 6000 from the aspect of the vesicle-forming properties.

The “R³” and “R⁴” units are hydrocarbon groups having 1 to 4 carbon atoms. Since the hydroxyl groups at the ends, which cause stickiness, can be etherified, the “R³” and “R⁴” units can improve compatibility with skin and provide a good feel in use. Examples of hydrocarbon groups include methyl groups, ethyl groups, n-propyl groups, isopropyl groups, n-butyl groups, sec-butyl groups, tert-butyl group and groups that are mixtures thereof, among which methyl groups and ethyl groups are preferable. The “R” and “R⁴” units may be the same or different, and vesicles may be formed by one type or by a combination of two or more types of block-type alkylene oxide derivatives in which “R³” and “R⁴” are the same or different.

In the alkylene oxide derivative represented by Formula (3) above, the “B” unit is a residue obtained by removing a hydroxyl group from a dimer diol. In this case, a dimer diol is a diol obtained by reducing a dimeric acid. In the case in which the “B” unit is another diol other than dimer diol, there are cases in which vesicles cannot be formed, or even if vesicles can be formed, the stability is insufficient.

The dimeric acid that serves as the raw material of the dimer diol is, for example, a dimer obtained by polymerizing an unsaturated fatty acid or a lower alcohol ester thereof. Specifically, it can be synthesized by a method of reacting an unsaturated fatty acid such as oleic acid, linolic acid or linoleic acid, or a lower alcohol ester thereof, by means of thermal polymerization, such as a Diels-Alder reaction, or another reaction method. Unreacted fatty acids may remain in the dimer acid that has been generated as long as they are within a range not compromising the effects of the present invention.

The dimer acid should preferably be obtained by dimerizing an unsaturated fatty acid having 12 to 24 carbon atoms, or a lower alcohol ester thereof. In this case, the “B” unit represents a dimer diol residue having 24 to 48 carbon atoms. Such unsaturated fatty acids include myristoleic acid, palmitoleic acid, oleic acid, elaidic acid, vaccenic acid, gadoleic acid, erucic acid, nervonic acid, linolic acid, linoleic acid, and esters thereof with lower alcohols having 1 to 3 carbon atoms, among which unsaturated fatty acids having 18 carbon atoms are preferred, and oleic acid, linolic acid or a lower alcohol ester thereof is more preferred. As the dimeric acid, a dimeric acid in which the dimerization is followed by hydrogenation of the remaining unsaturated double bonds may be used.

Animal oil-derived and vegetable oil-derived dimer diols are commercially available and can all be used in the present invention. However, those that are derived from vegetable oils are more preferred. Examples of such dimer diols include Sovermol 908 (manufactured by Cognis Japan, Ltd.), PRIPOL (registered trademark) 2033 (manufactured by Unigema, Ltd.), Pespol HP-1000 (manufactured by Toagosei Co., Ltd.), etc.

Though the block-type alkylene oxide derivatives represented by Formula (3) above are not particularly limited, they include, for example, POB(25) POE(34) dimethyl dimer diol ether, POB(25) POE(35) dimethyl dimer diol ether, POB(4) POE(13) dimethyl dimer diol ether, POB(5) POE(15) dimethyl dimer diol ether, POB(6) POE(18) dimethyl dimer diol ether, POB(7) POE(20) dimethyl dimer diol ether, POB(10) POE(24) dimethyl dimer diol ether, POB(10) POE(30) dimethyl dimer diol ether, POB(25) POE(52) dimethyl dimer diol ether, POB(18) POE(41) dimethyl dimer diol ether, POB(18) POE(41) diethyl dimer diol ether, POB(18) POE(41) dipropyl dimer diol ether, POB(18) POE(41) dibutyl dimer diol ether, POB(11) POE(30) dimethyl dimer diol ether, POB(15) POE(45) dimethyl dimer diol ether, POB(18) POE(50) dimethyl dimer diol ether, POB(21) POE(56) dimethyl dimer diol ether, POB(12) POE(50) dimethyl dimer diol ether, POB(18) POE(61) dimethyl dimer diol ether, POB(3) POE(40) dimethyl dimer diol ether, POB(6) POE(82) dimethyl dimer diol ether, POB(40) POE(120) dimethyl dimer diol ether, POB(100) POE(40) dimethyl dimer diol ether, POE(35) POP(30) dimethyl dimer diol ether, POE(52) POP(30) dimethyl dimer diol ether, etc. In this case, the number of moles of POE, POP and POB added are, respectively, the overall number of moles added to the molecules, i.e., represented by the values r¹+r² and s¹+s².

These block-type alkylene oxide derivatives can be manufactured by a known method. For example, the block-type alkylene oxide derivatives can be obtained by addition-polymerizing ethylene oxides and alkylene oxides having 3 or 4 carbon atoms onto compounds having hydroxyl groups, then having the alkyl halides undergo an ether reaction in the presence of an alkaline catalyst.

### (Sugar Fatty Acid Esters)

The sugar fatty acid esters include, for example, sucrose fatty acid esters, maltitol fatty acid esters, trehalose fatty acid esters, etc.

Though the number of hydroxyl groups substituted by fatty acids is not particularly limited, monoesters, diesters and triesters are preferred, monoesters and diesters are more preferred, and monoesters are further preferred.

The constituent fatty acids in the sugar fatty acid esters are preferably saturated or unsaturated fatty acids having 12 to 22 carbon atoms, preferably having linear chains or branches. These fatty acids include lauric acid, myristic acid, palmitic acid, stearic acid, isostearic acid, arachidic acid, behenic acid, tetradecenic acid, hexadecenic acid, octadecenic acid, octadecadienoic acid, eicosenoic acid, eicosatetraenoic acid, docosenoic acid, octadecatrienoic acid, etc., among which stearic acid is preferred. In the case of diesters, the two fatty acids may be different.

### (Polyoxyethylene Hydrogenated Castor Oil Derivatives)

As the polyoxyethylene hydrogenated castor oil derivatives, it is preferable to use compounds indicated by Formula (4) below.

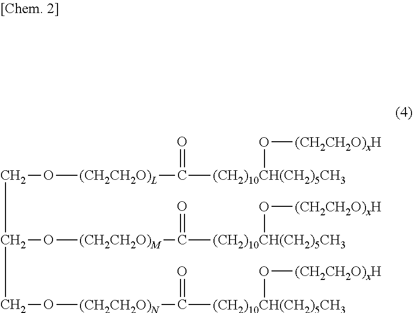

In Formula (4) above, L+M+N+X+Y+Z indicates the average number E of moles of ethylene oxides added, such that 10≤E≤20.

That is, as the polyoxyethylene hydrogenated castor oil derivative, it is possible to use a derivative in which the average number E of moles of ethylene oxide (EO) added is 10 to 20. If the average number of moles of EO added is less than 10, then vesicle particles will not spontaneously form in the water phase, and the emulsion composition of the present invention cannot be obtained. Additionally, if the number is greater than 20, then sufficient emulsification cannot be achieved while forming vesicle particles, and there is a sense of sliminess and the compatibility with skin is poor. Thus, satisfactory results cannot be obtained in terms of the texture. Additionally, the HLB value of the polyoxyethylene hydrogenated castor oil derivative blended into the oil-in-water emulsion cosmetic of the present invention is preferably 11 or lower.

### (Acyl Amino Acid Metal Salts)

The acyl amino acid metal salts are preferably acyl amino acid metal salts having 12 to 22 carbon atoms. Such acyl amino acid metal salts include, for example, sodium N-lauroyl-L-glutamate, sodium N-stearoyl-L-glutamate, sodium di(N-lauroylglutamyl)lysine, etc.

### (Phospholipids)

The phospholipids include, for example, egg yolk phospholipids, soy phospholipids and hydrogenates thereof, sphingophospholipids such as sphingomyelin, glycerophospholipids such as lecithin, etc.

As component (A) blended into the cosmetic according to the present invention, it is possible to use one type or a combination of two or more types of substances selected from among the above-mentioned vesicle-forming amphiphilic substances. In particular, it is preferable to use one or more types of substances selected from among the polyoxyalkylene-modified silicones represented by Formula (1) above and the polyoxyethylene hydrogenated castor oil derivatives represented by Formula (4) above, and more preferable to use one or more types of polyoxyalkylene-modified silicones represented by Formula (1) above.

Though the blended amount of component (A) is not particularly limited as long as vesicles can be formed, the blended amount is, for example, 0.1% to 5.0% by mass, preferably 0.3% to 3.0% by mass, and more preferably 0.8% to 2.0% by mass relative to the overall amount of the cosmetic. If the blended amount is less than 0.1% by mass, then there are cases in which sufficient emulsifying power cannot be obtained, and if the blended amount exceeds 5.0% by mass, then there are cases in which the feeling in use is made worse, such as becoming sticky.

<(B) Oil Component Containing UV Absorbing Agent Other than Ethylhexyl Methoxycinnamate>

The (B) oil component (hereinafter sometimes referred to simply as “component (B)”) blended into the cosmetic according to the present invention refers to an oil component essentially containing a UV absorbing agent other than ethylhexyl methoxycinnamate. From the aspect of obtaining UV protection ability by a UV absorbing agent other than ethylhexyl methoxycinnamate, the cosmetic of the present invention includes embodiments in which, when containing ethylhexyl methoxycinnamate, the amount is 1% by mass or less, and embodiments in which ethylhexyl methoxycinnamate is substantially not contained.

Though the UV absorbing agents other than ethylhexyl methoxycinnamate are not particularly limited, a type that is normally blended into cosmetics can be used. These include, for example, benzoic acid derivatives such as ethyl para-aminobenzoate (PABA), ethyl-dihydroxypropyl PABA, ethylhexyl-dimethyl PABA, glyceryl PABA, PEG-25-PABA and diethylaminohydroxybenzoyl hexyl benzoate; salicylic acid derivatives such as homosalate, ethylhexyl salicylate or octyl salicylate, dipropylene glycol salicylate and TEA salicylate; cinnamic acid derivatives such as isopropyl methoxycinnamate, isoamyl methoxycinnamate, cinnoxate, DEA methoxycinnamate, diisopropyl methyl cinnamate, glyceryl-ethylhexanoate-dimethoxycinnamate and di-(2-ethylhexyl)-4′-methoxybenzalmalonate; dibenzoylmethane derivatives such as 4-tert-butyl-4′-methoxydibenzoylmethane; β,β-diphenyl acrylate derivatives such as octocrylene; benzophenone derivatives such as benzophenone-1, benzophenone-2, benzophenone-3 or oxybenzone, benzophenone-4, benzophenone-5, benzophenone-6, benzophenone-8, benzophenone-9 and benzophenone-12; benzylidene camphor derivatives such as 3-benzylidene camphor, 4-methylbenzylidene camphor, benzylidene camphor sulfonic acid, camphor benzalkonium methosulfate, terephthalylidene dicamphor sulfonic acid and polyacrylamide methylbenzylidene camphor; phenyl benzimidazole derivatives such as phenyl benzimidazole sulfonic acid and disodium phenyl dibenzimidazole tetrasulfonate; triazine derivatives such as bis-ethylhexyloxyphenol methoxyphenyl triazine, ethylhexyl triazone, diethylhexyl butamido triazone, 2,4,6-tris(diisobutyl-4′-aminobenzalmalonate)-s-triazine and 2,4,6-tris[4-(2-ethylhexyloxycarbonyl)anilino]-1,3,5-triazine; phenylbenzotriazole derivatives such as drometrizole trisiloxane and methylene bis(benzotriazolyl tetramethylbutylphenol); anthranil derivatives such as menthyl anthranilate; imidazoline derivatives such as ethylhexyl dimethoxy benzylidene dioxoimidazoline propionate; benzalmalonate derivatives such as polyorganosiloxanes having benzalmalonate functional groups; 4,4-diarylbutadiene derivatives such as 1,1-dicarboxy(2,2′-dimethylpropyl)-4,4-diphenylbutadiene; etc. The UV absorbing agents used in the present invention may be blended as a single type or as a combination of two or more types.

Among the above, the UV absorbing agent in the present invention preferably essentially includes one or more type selected from among salicylic acid derivatives and D3,3-diphenyl acrylate derivatives. Furthermore, it preferably essentially contains octocrylene, homosalate and octyl salicylate.

The blended amount of the UV absorbing agent blended into the cosmetic of the present invention is 1% to 40% by mass, preferably 3% to 30% by mass, and more preferably 5% to 25% by mass relative to the overall amount of the cosmetic. If the blended amount of the UV absorbing agent is less than 1% by mass, then sufficient UV protection effects are difficult to obtain, and even if more than 40% by mass is blended, an increase in UV protection effects commensurate with the blended amount cannot be expected, and the stability and properties in use become worse.

One or more oils selected from among hydrocarbon oils, ester oils and silicone oils can be further blended into the (B) oil component in the present invention.

Specific examples of hydrocarbon oils include isododecane, isohexadecane, hydrogeneated polydecene, isoparaffin, liquid paraffin, ozokerite, squalane, pristane, paraffin, ceresin, squalene, vaseline, microcrystalline wax, etc.

Specific examples of ester oils include isopropyl myristate, cetyl ethylhexanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentylglycol dicaprate, diisostearyl malate, glycerin di-2-heptylundecanoate, trimethylolpropane tri-2-ethylhexanoate, trimethylolpropane triisostearate, glycerin triisostearate, pentaerythrityl tetraethylhexanozte, triethylhexanoin (glyceryl tri-2-ethylhexanoate), cetyl 2-ethylhexanoate, 2-ethylhexyl palmitate, glycerin trimyristate, tri-2-heptylundecanoic acid glyceride, castor oil fatty acid methyl ester, oleyl oleate, cetostearyl alcohol, acetoglyceride, 2-heptylundecyl palmitate, diisobutyl adipate, 2-octyldodecyl N-lauroyl-L-glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethlhexyl succinate, polypropylene glycol dipivalate, ethyl acetate, butyl acetate, amyl acetate, triethyl citrate, etc.

Specific examples of silicone oils include linear polysiloxanes (e.g., dimethyl polysiloxane, methylphenyl polysiloxane, diphenyl polysiloxane, etc.), cyclic polysiloxanes (e.g., octamethyl cyclotetrasiloxane, decamethyl cyclopentasiloxane, dodecamethyl cyclohexasiloxane, etc.), silicone resins forming three-dimensional network structures, silicone rubber, various types of modified polysiloxanes (amino-modified polysiloxane, polyether-modified polysiloxane, alkyl-modified polysiloxane, fluorine-modified polysiloxane, etc.), acrylic silicones, etc.

The blended amount of the (B) oil component is not particularly limited as long as it is an amount that is normally used when blending pigments into an oil phase, and for example, may be 1% to 40% by mass relative to the overall amount of the cosmetic. If the blended amount of the (B) oil component exceeds 40% by mass, then there is a tendency for the stability and the properties in use to become lower.

The (B) oil component in the present invention only needs to include at least the UV absorbing agent other than ethylhexyl methoxycinnamate. Thus, the cosmetic of the present invention includes embodiments containing only the UV absorbing agent as the oil component.

In the cosmetic according to the present invention, from the aspect of further increasing the vibration stability, when a low-molecular-weight oil component is blended as the (B) oil component, the percentage occupied by the low-molecular-weight oil relative to the total blended amount of the oil component other than the UV absorbing agent should preferably be 50% or less in terms of mass. Since a low-molecular-weight oil component does not need to be blended as the (B) oil component in the present invention, the range of percentages occupied by the low-molecular-weight oil component relative to the total blended amount of the oil component other than the UV absorbing agent is 0% to 50%.

In the present invention, the low-molecular-weight oil component refers to an oil component for which the volatilization rate at 25° C. is 30% or higher in terms of the weight change per hour. In this case, the volatilization rate refers to the value of the weight change per hour when measured under 25° C. conditions by means of a gravimetric method when filter paper is placed on a glass Petri dish and approximately 0.2 g of the sample is dripped thereon. Specific examples of low-molecular-weight oil components include isododecane, low-viscosity volatile silicones (low-melting-point dimethicones) having an average degree of polymerization of less than 650, etc. Commercially available products that are low-molecular-weight oil components include Creasil ID CG (manufactured by Shima Trading Co., Ltd.), KF-96L-1.5CS (manufactured by Shin-Etsu Chemical Co., Ltd.), etc. The volatilization rate of Creasil ID CG is 90% or higher, and the volatilization rate of KF-96L-1.5CS is approximately 50%.

### <(C) Amino Acid Surface-Treated Pigment>

The (C) amino acid surface-treated pigment (hereinafter sometimes referred to simply as “component (C)”) blended into the cosmetic according to the present invention refers to an inorganic pigment with surfaces that have been hydrophobically treated by a treatment agent containing an amino acid. Specific examples include iron oxide (Bengal red), iron titanate, γ-iron oxide, yellow iron oxide, ocher, black iron oxide, carbon black, low-order titanium oxide, mango violet, cobalt violet, chromium oxide, chromium hydroxide, cobalt titanate, ultramarine blue, Prussian blue, etc. Among the above, as the pigment in the present invention, it is preferable to use a pigment-grade iron oxide such as yellow iron oxide, red iron oxide or black iron oxide, a pigment-grade titanium oxide, etc. In this case, pigment grade refers to the average particle size being 100 nm to 1 μm.

The amino acid used in the hydrophobic surface treatment of the pigments in the present invention should preferably be an amino acid that has been acylated by means of a saturated fatty acid, or a salt thereof. The expression “an amino acid that has been acylated by means of a saturated fatty acid, or a salt thereof” refers to a compound in which acyl groups, preferably saturated fatty acids having 12 to 22 carbon atoms, are condensed onto the amino groups of an amino acid, or salts thereof. The “amino acid” is preferably glutamic acid or aspartic acid. Examples of the “acyl groups” include stearoyl groups, lauroyl groups, etc. The “salts” may be selected from among alkali metal salts such as sodium and potassium, alkaline earth metal salts, etc., among which sodium salts are preferred. Specific examples of acylated amino acids include disodium stearoyl glutamate, sodium lauroyl glutamate and sodium lauroyl aspartate.

Examples of the amino acid used for the hydrophobic surface treatment of the pigment in the present invention include N-acyl glutamic acid, N-acyl aspartic acid, N-acyl lysine, etc.

The (C) pigment used in the cosmetic of the present invention is preferably surface-treated by a treatment agent including an amino acid selected from among acylated glutamic acid and acylated aspartic acid.

Commercially available products that are pigment-grade iron oxides that have been hydrophobically treated with amino acids include NHS-Yellow-LL-100P (manufactured by Miyoshi Kasei Industry Co., Ltd.), ASI-Yellow-LL-100P (manufactured by Daito Kasei Kogyo Co., Ltd.) and ASL-Yellow-LL-100P (manufactured by Daito Kasei Kogyo Co., Ltd.), which are yellow iron oxides; NHS-Black BL-100P (manufactured by Miyoshi Kasei Industry Co., Ltd.), ASI-Black BL-100P (manufactured by Daito Kasei Kogyo Co., Ltd.) and ASL-Black BL-100P (manufactured by Daito Kasei Kogyo Co., Ltd.), which are black iron oxides; NHS-Red R516PS (manufactured by Miyoshi Kasei Industry Co., Ltd.), ASI-Red R516PS (manufactured by Daito Kasei Kogyo Co., Ltd.) and ASL-Red R516PS (manufactured by Daito Kasei Kogyo Co., Ltd.), which are red iron oxides; NHS-titanium CR-50 (manufactured by Miyoshi Kasei Industry Co., Ltd.), ASI-titanium CR-50 (manufactured by Daito Kasei Kogyo Co., Ltd.) and ASL-titanium CR-50 (manufactured by Daito Kasei Kogyo Co., Ltd.), which are titanium oxides; etc.

The blended amount of component (C) is not particularly limited as long as the desired colors are obtained, but should normally be 1% by mass or more relative to the overall amount of the cosmetic, for example, 1% to 30% by mass, and preferably 1% to 20% by mass. If the blended amount is less than 1% by mass, then sufficient colors cannot be obtained, and if 30% by mass is exceeded, then there is a tendency for the stability to become poor.

The cosmetic according to the present invention is a powder-in-oil-in-water type composition in which the (C) pigment is dispersed in oil droplets, which constitute the internal phase.

In the cosmetic according to the present invention, the oil phase should preferably be 1% to 50% by mass relative to the overall amount of the cosmetic.

A (D) aqueous component (hereinafter sometimes referred to simply as “component (D)”) may be blended into the cosmetic according to the present invention. The (D) aqueous component in the present invention refers to a component mainly composed of water, ethanol (ethyl alcohol), or a polyhydric alcohol such as dipropylene glycol, 1,3-butylene glycol or glycerin.

In the present invention, as the (D) aqueous component, one or more types selected from among monohydric alcohols and dihydric alcohols are preferably used.

The monohydric alcohols are not particularly limited as long as they are normally used in cosmetics, and examples include ethyl alcohol, normal propyl alcohol, isopropyl alcohol, etc., among which ethyl alcohol is preferred.

The dihydric alcohols are not particularly limited as long as they are normally used in cosmetics, and examples include 1,3-butylene glycol, dipropylene glycol, etc., among which dipropylene glycol is preferred.

When only a monohydric alcohol is used as the (D) aqueous component, the blended amount thereof is 1% to 15% by mass relative to the overall amount of the cosmetic, and when only a dihydric alcohol is used, the blended amount thereof is 1% to 20% by mass relative to the overall amount of the cosmetic. Additionally, when using a combination of a monohydric alcohol and a dihydric alcohol, the total blended amount thereof is 1% to 45% by mass, preferably 1% to 35% by mass, relative to the overall amount of the cosmetic. More preferably, they should be blended such that the upper limits of the concentrations of the monohydric alcohol and the dihydric alcohol satisfy Expression (5) below.

Monohydric alcohol concentration (%) in water phase/15+dihydric alcohol concentration (% by mass) in water phase/20≤1 (5)

When, as the (D) aqueous component, the blended amount of a monohydric alcohol, the blended amount of a dihydric alcohol, or the total blended amount of a monohydric alcohol and a dihydric alcohol is less than 1% by mass, there are cases in which vesicles are not generated or the structure is disturbed and the emulsification becomes weak. Additionally, in the case in which the blended amount of a monohydric alcohol exceeds 15% by mass or the blended amount of a dihydric alcohol exceeds 20% by mass, or further, in the case in which the blending ratio of the monohydric alcohol and the dihydric alcohol is outside the range in Expression (5) above, or even if within the range in Expression (5) above, when the total blended amount exceeds 45% by mass, there are cases in which the vesicle films become too soft or the vesicles transition to micelles, so that stability improvement effects cannot be obtained.

An (E) ionic surfactant (hereinafter also referred to simply as “component (E)”) may further be blended into the cosmetic according to the present invention. When a vesicle-forming amphiphilic substance is used as the emulsifier in the present invention, the stability of the vesicles can be further improved by blending an ionic surfactant into the water phase.

The (E) ionic surfactant blended into the cosmetic according to the present invention merely needs to be of a type that is normally used in cosmetics, and refers to a surfactant other than component (A) above, that also exhibits ionicity.

As the (E) ionic surfactant blended into the cosmetic according to the present invention, an anionic surfactant should preferably be used. Among such surfactants, sulfonic acid salt-type anionic surfactants are preferred. As the sulfonic acid salt-type anionic surfactant, it is preferable to use one selected from among sulfosuccinic acid diester salts, alkylallyl sulfonic acid salts, alkyl ether sulfonic acid salts, sulfosuccinic acid ester salts, acyl methyl taurine salts, acyl taurine salts, potassium cetyl phosphate, potassium cocoyl glutamate, etc. Among the above, it is preferable to use one selected from among acyl methyl taurine salts, potassium cetyl phosphate and potassium cocoyl glutamate, and more preferable to use N-acyl methyl taurine salts.

Furthermore, among the N-acyl methyl taurine salts represented by Formula (6) below, N-stearoyl-N-methyl taurine salts are preferred.

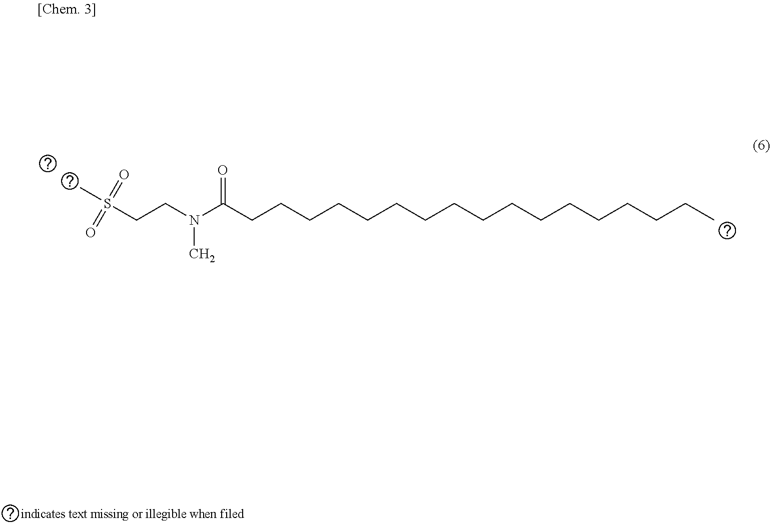 [SMILES: CCCCCCCCCCCCCCCCCC(=O)N(C)CCS(C)(=O)=O]

The blended amount of component (E) should preferably be 0.01% to 1% by mass, more preferably 0.01% to 0.1% by mass, and even more preferably 0.01% to 0.06% by mass relative to the overall amount of the cosmetic. If the blended amount is less than 0.01% by mass, then there are cases in which sufficient vesicle stability improvement effects cannot be obtained, and if 1% by mass is exceeded, then there are cases in which the vesicles solubilize.

Additionally, the blending ratio between component (A) and the (E) ionic surfactant should preferably be 1:0.01 to 1:0.06 in terms of the ratio by mass.

In general, whiteners are also blended into sunscreen cosmetics. However, it is known that there is normally a tendency for whiteners of the salt type to have poor stability when blended into a water phase that is an external phase. The cosmetic according to the present invention has sufficient emulsifying power, and therefore provides the effect of having exceptional vibration stability even when a salt-type whitener is blended into the water phase.

The (F) whitener (hereinafter sometime referred to simply as “component (F)”) blended into the cosmetic according to the present invention is not particularly limited as long as it is of a type that is normally blended into cosmetics. Specific examples include L-ascorbic acid and derivatives thereof, tranexamic acid and derivatives thereof, alkoxysalicylic acid and derivatives thereof, glycyrrhizic acid and derivatives thereof, nicotinic acid and derivatives thereof, etc. In the cosmetic according to the present invention, the aforementioned agents may be blended as a single type or as a combination of two or more types.

As the L-ascorbic acid derivatives, it is preferable to use L-ascorbic acid monoalkyl esters such as L-ascorbic acid monostearate, L-ascorbic acid monopalmitate and L-ascorbic acid mono-oleate; L-ascorbic acid monoesters such as L-ascorbic acid monophosphoric acid ester and L-ascorbic acid-2-sulfuric acid ester; L-ascorbic acid dialkyl esters such as L-ascorbic acid distearate, L-ascorbic acid dipalmitate and L-ascorbic acid dioleate; L-ascorbic acid trialkyl esters such as L-ascorbic acid tristearate, L-ascorbic acid tripalmitate and L-ascorbic acid trioleate; L-ascorbic acid triesters such as L-ascorbic acid triphosphoric acid esters; L-ascorbic acid glucosides such as L-ascorbic acid 2-glucoside; etc. In the present invention, L-ascorbic acid, L-ascorbic acid phosphoric acid ester, L-ascorbic acid 2-sulfuric acid ester, L-ascorbic acid 2-glucoside and salts thereof are preferably used.

As the tranexamic acid derivatives, it is preferable to use tranexamic acid dimers (for example, hydrochloric acid trans-4-(trans-aminomethyl cyclohexanecarbonyl) aminomethyl cyclohexane carboxylic acid, etc.), esters of tranexamic acid with hydroquinone (for example, 4-(trans-aminomethyl cyclohexane carboxylic acid 4′-hydroxyphenyl ester, etc.), esters of tranexamic acid with gentisic acid esters (for example, 2-(trans-4-aminomethyl cyclohexylcarbonyloxy)-5-hydroxybenzoic acid, etc.), tranexamic acid amides (for example, trans-4-aminomethyl cyclohexane carboxylic acid methyl amide, trans-4-(p-methoxybenzoyl)aminomethyl cyclohexane carboxylic acid, trans-4-guanidinomethyl cyclohexane carboxylic acid, etc.), and the like. In the present invention, tranexamie acid and salts thereof are preferably used.

In the alkoxysalicylic acid derivatives, the hydrogen atom at one of the 3-position, the 4-position or the 5-position on the salicylic acid is substituted with an alkoxy group, the alkoxy group that is the substituent group preferably being one of a methoxy group, an ethoxy group, a propoxy group, an isopropoxy group, a butoxy group and an isobutoxy group, and more preferably, a methoxy group or an ethoxy group. Examples of specific compounds include 3-methoxysalicylic acid, 3-ethoxysalicylic acid, 4-methoxysalicylic acid, 4-ethoxysalicylic acid, 4-propoxysalicylic acid, 4-isopropoxysalicylic acid, 4-butoxysalicylic acid, 5-methoxysalicylic acid, 5-ethoxysalicylic acid, 5-propoxysalicylic acid, etc. In the present invention, methoxysalicylic acid and salts thereof (methoxysalicylic acid potassium salts) are preferably used.

As the glycyrrhizic acid derivatives, there are glycyrrhizic acid salts, esters of glycyrrhizic acid with higher alcohols, etc. In the present invention, glycyrrhizic acid and salts thereof (glycyrrhizic acid potassium salts, glycyrrhizic acid monoammonium salts, etc.) are preferably used.

Although the salts of the above-mentioned agents are not particularly limited, they may be, for example, alkali metal salts or alkaline earth metal salts such as sodium salts, potassium salts and calcium salts, as well as ammonium salts, amino acid salts, etc.

As the nicotinic acid and derivatives thereof, there are nicotinic acid, benzyl nicotinate, nicotinic acid amide, dl-α-tocopherol nicotinate, etc. In the present invention, nicotinic acid amide is preferably used.

The blended amount of the (F) whitener should preferably be 0.05% to 10% by mass, more preferably 0.1% to 7% by mass, and even more preferably 0.5% to 5% by mass relative to the overall amount of the cosmetic. If the blended amount is less than 0.05% by mass, then there are cases in which sufficient efficacy cannot be obtained, and if 10% by mass is exceeded, then the stability and properties in use tend to become worse.

The (F) whitener in the present invention may be dissolved or dispersed in the water phase together with other aqueous components, or may be encapsulated inside the vesicles by being dissolved or dispersed in the water phase during the vesicle formation process.

One or more dispersants may be blended into the cosmetic according to the present invention. Specific examples of dispersants include sorbitan sequiisostearate, isostearic acid, palmitic acid, polyhydroxystearic acid, etc. Among these, sorbitan sesquiisostearate and isostearic acid are particularly preferable examples, and either one or both of these may be blended.

The dispersant is an optional blended component in the cosmetic of the present invention and thus is not necessarily required to be blended therein. However, if blended, it should be blended in an amount such that the effects of the blending can be observed, and within limits such that the blended amount does not become excessive and problems such as degrading the feeling in use are not observed. The blended amount of the dispersant in the cosmetic according to the present invention is preferably approximately 0.01% to 1% by mass relative to the overall amount of the cosmetic.

The water blended into the cosmetic according to the present invention is selected, as needed, from among ion-exchanged water, purified water, tap water, natural spring water, etc. The blended amount is the remaining amount (percentage by mass relative to the overall amount of the cosmetic) relative to the sum of the essential components of the present invention and the other optional blended components. In general, the amount should preferably be approximately 30% to 70% by mass relative to the overall amount of the cosmetic.

The oil-in-water emulsion cosmetic according to the present invention may contain, aside from the above-mentioned components, other optional additive components that are normally used in external skin-care preparations such as cosmetic products and pharmaceutical products, within a range not compromising the purpose and effects of the present invention. For example, oils/fats, waxes, higher fatty acids, higher alcohols, oil phase thickeners, surfactants, UV scattering agents, water-soluble UV absorbing agents, chelating agents, lower alcohols, polyhydric alcohols, pH adjusters, antioxidants, powder components, fragrances, etc. may be appropriately blended as needed. However, the optional components are not limited to these examples.

### <Vesicle Emulsion>

The oil-in-water emulsion cosmetic that is an embodiment of the present invention is characterized by being emulsified by vesicles formed from component (A). In this case, “vesicles” refer to spherical enclosed bodies having bimolecular film (lamellar liquid crystal) structures formed from amphiphilic block copolymers, such that water-soluble components can be held inside the enclosed bodies and oil-based components can be held inside the bimolecular films, contributing to emulsification. The vesicle-forming amphiphilic substance forms vesicles in the water phase, then the water phase is emulsified with the oil phase in the presence of the vesicles, resulting in a water phase/vesicle phase/oil phase three-phase structure in which the vesicles are adhered to the surfaces of oil droplets, and the oil droplets stably disperse in the water phase. In the present specification, this emulsification method will be referred to as “vesicle emulsification”.

The vesicles can be formed by a conventional method. For example, vesicles comprising the vesicle-forming amphiphilic substance are formed in the water phase by mixing and stirring the aqueous component with the vesicle-forming amphiphilic substance. During vesicle formation, water-soluble components that are normally used in cosmetic products may be blended with the aqueous component in an amount within a range not compromising the stability of the vesicles. Though the average particle size of the vesicles is not particularly limited, they should normally be approximately 30 nm to 150 nm.

Additionally, the vesicles according to the present invention can be produced, by a conventional method, in a form encapsulating water-soluble components inside the vesicles or in a form holding oil-based components inside the biomolecular films of the vesicles. Specifically, the vesicles according to the present invention may be produced as vesicles encapsulating water-soluble agents such as whiteners by dissolving or dispersing the agents in the water phase. Additionally, the vesicles according to the present invention can be produced as vesicles holding oil-soluble components such as fragrances in the bimolecular films of the vesicles by adding and mixing the oil-soluble components during the step of mixing the vesicle-forming amphiphilic substance with the aqueous component.

The vesicles according to the present invention can be produced as aqueous vesicle dispersions by well-mixing the vesicle-forming amphiphilic substance with the aqueous component, then dripping the mixed solution into a water phase containing the other water-soluble components while stirring. The mixture state of the vesicle-forming amphiphilic substance and the aqueous component need only be such as to be able to confirm that the mixed solution is transparent and in a single-phase state. This can be achieved, for example, by mixing for 1 to 30 minutes at a temperature from room temperature to 90° C. Due to this method, vesicle particles for which the average particle size measured by dynamic light scattering is 30 to 150 nm are obtained. The stirring device used for stirring is not particularly limited, and for example, a homomixer, a dispersion mixer or the like may be used.

In the present invention, the vesicle particles formed in the water phase can be uniformly dispersed in the water phase by applying strong shear with the aforementioned homomixer or the like, thereby making the particle size sufficiently small. Though the degree of the strong shear is not particularly limited, it should normally be for approximately 5 minutes at 4000 to 12000 rpm by a homomixer.

In the oil-in-water emulsion cosmetic emulsified by vesicles, which is an embodiment of the present invention, when further blending an (E) ionic surfactant, the emulsification preferably involves forming the vesicle particles in the water phase, adding an ionic surfactant to this vesicle-containing water phase, then adding and emulsifying the oil-based components.

The oil-in-water emulsion cosmetic obtained by emulsification by means of vesicles, which is an embodiment of the present invention, can be produced by a conventional method. For example, the method may include a vesicle formation step of forming vesicles by mixing the aqueous component and the vesicle-forming amphiphilic substance necessary for vesicle formation, and in some cases, a step of adding an ionic surfactant to the aqueous vesicle dispersion obtained by the aforementioned step, and an emulsification step of emulsification by adding the oil-based components, which have been separately mixed and dissolved, to the water phase solution obtained in the aforementioned step, while stirring and applying a shear force.

In the aforementioned vesicle formation step, a vesicle dispersion in which vesicles are dispersed in the water phase can be obtained by dissolving the aqueous component and the vesicle-forming amphiphilic substance necessary for vesicle formation in advance, and mixing the dissolved substance into the remaining water and other water-soluble components, or a vesicle dispersion in which vesicles are dispersed in the water phase can be obtained by mixing and stirring the vesicle-forming amphiphilic substance into a water phase including the aqueous component, water and the other water-soluble components.

### <Nanodisk Emulsion>

In the oil-in-water emulsion cosmetic according to another embodiment of the present invention, a nanodisk emulsion can be realized by using a polyoxyalkylene-modified silicone represented by the above Formula (1) as the component (A), using an aqueous component selected from among monohydric alcohols and dihydric alcohols as the component (D), and using an anionic surfactant as the component (E).

Specifically, an emulsion cosmetic having a water phase/nanodisk phase/oil phase three-phase structure in which nanodisks formed by structurally changing vesicles are adhered to oil-water interfaces can be obtained by using a polyoxyalkylene-modified silicone (component (A)) represented by the above Formula (1) and an aqueous component (component (D)) selected from among monohydric alcohols and dihydric alcohols to form vesicles, adding an anionic surfactant (component (E)) and oil-based components to the vesicle-containing aqueous solution, and stirring and dispersing the mixture. In the present specification, this type of emulsion by nanodisks is referred to as “nanodisk emulsion”. Cosmetics obtained by nanodisk emulsion have extremely strong emulsifying power and have excellent vibration stability.

In this case, the “nanodisks” refer to flat plate-shaped lamellar liquid crystal enclosed bodies having the vesicles (lamellar liquid-crystal spherical enclosed bodies) formed by the amphiphilic substances as precursors, wherein water-soluble components are not encapsulated within the enclosed bodies and edge portions have lipophilic groups. The nanodisks exist as vesicles, which are the precursors, in a composition not containing oils, and the vesicles undergo a structural change (hereinafter also referred to as a “transition”) to nanodisks when an oil is added and emulsification is performed. The nanodisks formed in the present invention are obtained by adding an anionic surfactant and an oil component to an aqueous vesicle dispersion in which vesicles are formed by mixing an aqueous component selected from among monohydric alcohols and dihydric alcohols with a polyoxyalkylene-modified silicone, and dispersing the vesicles while applying a strong stirring force. The nanodisks exist in a state of adsorption to the oil-water interfaces in the emulsion state, and contribute to emulsion stability. In the nanodisks in the present invention, the amphiphilic substance forming the vesicles is a silicone-based surfactant. Thus, the nanodisks are also referred to as “silicone nanodisks”. The nanodisks have diameters, on the major axes, of 20 nm to 1000 nm.

According to the present invention, in the embodiment of the oil-in-water emulsion cosmetic obtained by nanodisk emulsion, water-soluble components are not encapsulated inside the vesicles. In this case, the vesicles may be produced as vesicles in which the oil-soluble components, such as fragrances, are held in the bimolecular films of the vesicles by adding and mixing the oil-soluble components during the step of mixing the vesicle-forming amphiphilic substance with the aqueous component.

Though the surfaces of the spherical vesicles formed by the surfactant are entirely covered with hydrophilic groups, the nanodisks have lipophilic groups at their edge portions. Thus, the nanodisks are difficult to form in water. If monohydric and dihydric alcohols are present in the water, then the surfactant (polyether-modified silicone, etc.) is hydrophilized due to solvent effects, and as a result thereof, the transition from spherical vesicles to nanodisks is promoted.

Meanwhile, when dissolving a polyoxyalkylene-modified silicone such as PEG-12 dimethicone in alcohol, trihydric alcohols such as glycerin and polyhydric alcohols such as sorbitol tend to lipophilize the surfactant and inhibit the transition to nanodisks. Thus, in the case in which trihydric or higher alcohols are to be blended, the total amount of monohydric and dihydric alcohols should preferably be greater than the total amount of trihydric or higher polyhydric alcohols.

In order to realize stable nanodisk emulsion, an anionic surfactant must be used as the component (E). The anionic surfactant may be of any type that is normally used in cosmetics, and refers to a surfactant having anionic hydrophilic groups, such as those having carboxylic acid, sulfonic acid or phosphoric acid structures. Among the above, those having a Krafft point higher than room temperature are preferably used as the anionic surfactant. If the Krafft point of the anionic surfactant is lower than room temperature, the silicone-based surfactant and the anionic surfactant easily mix together and interact. Thus, the transition from vesicles to nanodisks tends to be hindered.

The anionic surfactant used for nanodisk emulsion is preferably selected from among acyl methyl taurine salts, potassium cetyl phosphate and potassium cocoyl glutamate, and more preferably, N-acyl methyl taurine is used. Additionally, among the N-acyl methyl taurine salts represented by Formula (6) below, N-stearoyl-N-methyl taurine salt is particularly preferred.

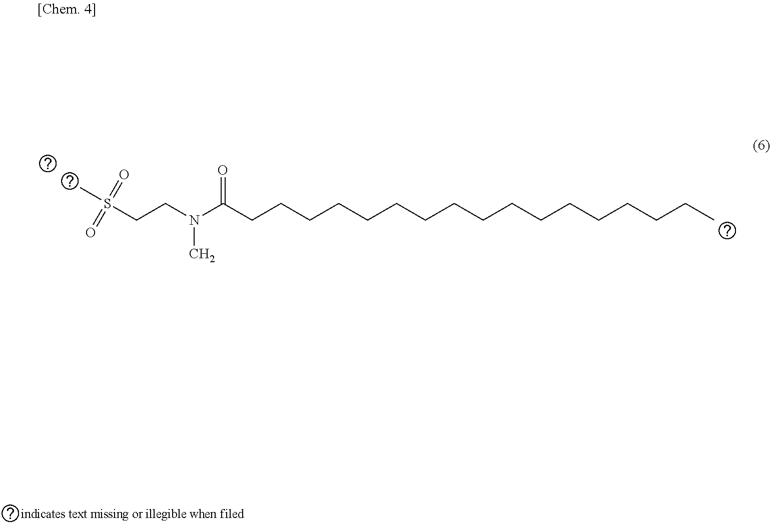 [SMILES: CCCCCCCCCCCCCCCCCC(=O)N(C)CCS(C)(=O)=O]

The oil-in-water emulsion cosmetic obtained by nanodisk emulsion, which is another embodiment of the present invention, can be produced by a conventional method. For example, an aqueous vesicle dispersion is obtained by forming vesicle particles by dripping the aqueous component into a polyoxyalkylene-modified silicone while stirring, an anionic surfactant and the separately mixed and dissolved oil-based components are added to this aqueous vesicle dispersion, and the vesicles are dispersed by a strong stirring force, causing the vesicles to transition to nanodisks, thereby forming a water phase/nanodisk phase/oil phase three-phase structure. Since oil droplets comprising the oil-based components are emulsified and dispersed in the water phase, and the nanodisks are localized at the surfaces of the oil droplet particles, the emulsion cosmetic has excellent emulsion stability and an excellent feel in use (wateriness, lack of stickiness). The stirring apparatus used for stirring is not particularly limited, and for example, a homomixer, a dispersion mixer, etc. may be used.

That is, the oil-in-water emulsion cosmetic produced by nanodisk emulsion according to the present invention is obtained by forming vesicle particles in the water phase, adding an anionic surfactant to the vesicle dispersion, then adding and emulsifying the oil-based components.

Therefore, the method for producing the oil-in-water emulsion cosmetic by nanodisk emulsion in the present invention comprises a vesicle-forming step of forming vesicles by mixing the aqueous component with the polyoxyalkylene-modified silicone, a step of obtaining a mixture by adding an anionic surfactant to the vesicle dispersion obtained in the vesicle-forming step, and an emulsification step of stirring the separately mixed and dissolved oil-based components into the mixture obtained by the previous step and allowing the mixture to emulsify while applying shear force.

In the vesicle-forming step, the aqueous component and the polyoxyalkylene-modified silicone can be dissolved in advance, then the dissolved substance can be mixed into the remaining water and other water-soluble components to obtain a vesicle dispersion in which the vesicles are dispersed in the water phase, or the polyoxyalkylene-modified silicone can be mixed and stirred into the water phase containing the aqueous component, water and the other water-soluble components to obtain a vesicle dispersion in which the vesicles are dispersed in the water phase.

The cosmetic according to the present invention has the effect of providing the oil-in-water emulsion with a characteristic watery texture in use, and improving the compatibility with skin, the uniformity of the coating film, and the non-thickly-coated appearance. Additionally, a fresh feel in use is obtained despite being able to blend, into the vesicle-containing emulsion or the nanodisk-containing emulsion according to the present invention, oil components in an amount that cannot normally be blended into solubilized substances.

The cosmetic according to the present invention is preferably used in various formats such as creams, milky lotions, liquids, etc. The product form may be a makeup cosmetic in which a pigment is blended, such as a makeup base, a foundation, an eyeliner, an eyeshadow, etc.

### EXAMPLES

Though the present invention will be explained in further detail by providing examples below, the present invention is not limited in any way by these examples. Where not specially noted, the blended amounts are indicated in percentage by mass relative to the systems in which the relevant components are blended. Before specifically explaining the respective examples, the evaluation methods that were employed will be explained.

### 1. Vibration Stability

Resin tubes were filled with 30 ml of prepared samples and subjected to vibrations for 30 minutes at 10 Hz or higher. After being placed at rest, the emulsion separation and powder aggregation states of the samples were observed by eye and evaluated based on the criteria below.
A: Demulsification and powder aggregation were not observed.
B: Demulsification and powder aggregation were mostly not observed.
C: Demulsification and powder aggregation were observed.
D: Demulsification and powder aggregation were intensely observed.
   2. Makeup Effects (Compatibility with Skin, Uniformity of Coating Film, Non-Thickly-Coated Appearance)

Evaluations were performed by actual usage tests by ten expert panelists. Specifically, the makeup effects (compatibility with skin, uniformity of coating film, non-thickly-coated appearance) when the prepared samples were applied to the skin were evaluated in accordance with each of the criteria below.
A: Seven or more of the ten panelists felt that the effects were good.
B: Four to six of the ten panelists felt that the effects were good.
C: Three or fewer of the ten panelists felt that the effects were good.

### Examples 1-6 and Comparative Examples 1-3

The oil-in-water emulsion cosmetics having the compositions indicated in the table on the next page were prepared. Specifically, ethanol and an amphiphilic substance (polyoxyalkylene-modified silicone, polyoxyethylene-modified hydrogenated castor oil) for forming vesicles were mixed and stirred, then the other aqueous components were mixed to obtain a water phase solution, an ionic surfactant was added to the water phase solution, then an oil phase solution obtained by separately mixing the oil-based components and the powder components was mixed into the water phase solution while stirring, thereby obtaining the oil-in-water emulsion cosmetics of Examples 1 to 6 and Comparative Examples 1 to 3. The vibration stability and the makeup effects were evaluated for the prepared samples in accordance with the aforementioned evaluation methods. The results are indicated in the table.

TABLE 1

| | | | | | | | | Comp. | Comp. | Comp. |
|---|---|---|---|---|---|---|---|---|---|---|
| | | Ex. 1 | Ex. 2 | Ex. 3 | Ex. 4 | Ex. 5 | Ex. 6 | Ex. 1 | Ex. 2 | Ex. 3 |
| | 1,3-Butylene glycol | 8 | 8 | 8 | 8 | 8 | 8 | 8 | 8 | 8 |
| D | Ethanol | 8 | 8 | 8 | 8 | 8 | 8 | 8 | 8 | 8 |
| A | PEG-12 dimethicone | 1.6 | 1.6 | 1.6 | 1.6 | — | 1.6 | 1.6 | 1.6 | 1.6 |
| | PEG-10 hydrogenated castor oil | — | — | — | — | 2.5 | — | — | — | — |
| B | Diisopropyl sebacate | 8 | 8 | 8 | 8 | 8 | 8 | 8 | 8 | 8 |
| | Octocrylene | 5 | 5 | 5 | 5 | 5 | 5 | 5 | 10 | — |
| | Ethylhexyl salicylate | 7 | 7 | 7 | 7 | 7 | 7 | 7 | — | 10 |
| C | Silicone/amino acid-treated titanium | 4 | 4 | — | — | 4 | — | — | — | — |
| | oxide *1 | | | | | | | | | |
| | Silicone/amino acid-treated red iron | 0.8 | 0.8 | — | — | 0.8 | — | — | — | — |
| | oxide *2 | | | | | | | | | |
| | Silicone/amino acid-treated yellow | 3.2 | 3.2 | — | — | 3.2 | — | — | — | — |
| | iron oxide *3 | | | | | | | | | |
| | Silicone/amino acid-treated black | 0.1 | 0.1 | — | — | 0.1 | — | — | — | — |
| | iron oxide *4 | | | | | | | | | |
| | Amino acid-treated titanium oxide *5 | — | — | 4 | — | — | — | — | — | — |
| | Amino acid-treated red iron oxide *6 | — | — | 0.8 | — | — | — | — | — | — |
| | Amino acid-treated yellow iron oxide | — | — | 3.2 | — | — | — | — | — | — |
| | *7 | | | | | | | | | |
| | Amino acid-treated black iron oxide | — | — | 0.1 | — | — | — | — | — | — |
| | *8 | | | | | | | | | |
| | Amino acid-treated titanium oxide *9 | — | — | — | 4 | — | 4 | — | — | — |
| | Amino acid-treated red iron oxide | — | — | — | 0.8 | — | 0.8 | — | — | — |
| | *10 | | | | | | | | | |
| | Amino acid-treated yellow iron oxide | — | — | — | 3.2 | — | 3.2 | — | — | — |
| | *11 | | | | | | | | | |
| | Amino acid-treated black iron oxide | — | — | — | 0.1 | — | 0.1 | — | — | — |
| | *12 | | | | | | | | | |
| | Silicone-treated titanium oxide *13 | — | — | — | — | — | — | 4 | 4 | 4 |
| | Silicone-treated yellow iron oxide | — | — | — | — | — | — | 2 | 2 | 2 |
| | *14 | | | | | | | | | |
| | Silicone-treated red iron oxide *15 | — | — | — | — | — | — | 1 | 1 | 1 |
| | Silicone-treated black iron oxide *16 | — | — | — | — | — | — | 0.1 | 0.1 | 0.1 |
| | Sorbitan sesquiisotearate | 0.5 | 0.5 | 0.5 | 0.5 | 0.5 | 0.5 | 0.5 | 0.5 | 0.5 |
| | Isostearic acid | 0.2 | 0.2 | 0.2 | 0.2 | 0.2 | 0.2 | 0.2 | 0.2 | 0.2 |
| | Ion-exchanged water | 26.54 | 26.54 | 26.54 | 26.54 | 25.64 | 25.14 | 26.14 | 29.44 | 29.44 |
| | EDTA-3Na•2H2O | 0.05 | 0.05 | 0.05 | 0.05 | 0.05 | 0.05 | 0.05 | 0.05 | 0.05 |
| | Glycerin | 6 | 6 | 6 | 6 | 6 | 6 | 6 | 6 | 6 |
| | Phenoxyethanol | 0.5 | 0.5 | 0.5 | 0.5 | 0.5 | 0.5 | 0.5 | 0.5 | 0.5 |
| | (Dimethylacrylamide/sodium | 0.4 | 0.4 | 0.4 | 0.4 | 0.4 | 0.8 | 0.8 | 0.5 | 0.5 |
| | acryloyldimethyl taurate) | | | | | | | | | |
| | crosspolymer | | | | | | | | | |
| | Xanthan gum | 0.1 | 0.1 | 0.1 | 0.1 | 0.1 | 0.1 | 0.1 | 0.1 | 0.1 |
| | Ion-exchanged water | 10 | 10 | 10 | 10 | 10 | 10 | 10 | 10 | 10 |
| F | 4-Methoxy salicylic acid potassium | — | — | — | — | — | 1 | 1 | — | — |
| | salt | | | | | | | | | |
| E | Sodium stearoylmethyl taurate | 0.01 | 0.01 | 0.01 | 0.01 | 0.01 | 0.01 | 0.01 | 0.01 | 0.01 |
| | Silica/silicone-treated zinc oxide | 10 | — | — | — | 10 | — | 10 | 10 | 10 |
| | Silicone-treated zinc oxide | — | 10 | 10 | 10 | — | 10 | — | — | — |
| | Total | 100 | 100 | 100 | 100 | 100 | 100 | 100 | 100 | 100 |
| Evalu- | Vibration stability | A | A | A | A | B | A | C | C | C |
| ation | Compatibility with skin | A | A | A | A | A | A | A | A | A |
| | Uniformity | A | A | A | A | A | A | A | A | A |
| | Non-thickly-coated appearance | A | A | A | A | A | A | A | A | A |

*1: Silicone/amino acid (trimethyl methoxysilane, disodium stearoyl glutamate, aluminum hydroxide)-treated titanium oxide
*2: Silicone/amino acid (trimethyl methoxysilane, disodium stearoyl glutamate, aluminum hydroxide)-treated red iron oxide
*3: Silicone/amino acid (trimethyl methoxysilane, disodium stearoyl glutamate, aluminum hydroxide)-treated yellow iron oxide
*4: Silicone/amino acid (trimethyl methoxysilane, disodium stearoyl glutamate, aluminum hydroxide)-treated black iron oxide
*5: Amino acid/ester (isostearyl sebacate, disodium stearoyl glutamate, aluminum hydroxide)-treated titanium oxide
*6: Amino acid/ester (isostearyl sebacate, disodium stearoyl glutamate, aluminum hydroxide)-treated red iron oxide
*7: Amino acid/ester (isostearyl sebacate, disodium stearoyl glutamate, aluminum hydroxide)-treated yellow iron oxide
*8: Amino acid/ester (isostearyl sebacate, disodium stearoyl glutamate, aluminum hydroxide)-treated black iron oxide
*9: Amino acid (lysine, sodium lauroyl glutamate, magnesium chloride)-treated titanium oxide
*10: Amino acid (lysine, sodium lauroyl glutamate, magnesium chloride)-treated red iron oxide
*11: Amino acid (lysine, sodium lauroyl glutamate, magnesium chloride)-treated yellow iron oxide
*12: Amino acid (lysine, sodium lauroyl glutamate, magnesium chloride)-treated black iron oxide
*13: Tetramethyl tetrahydrogen cyclotetrasiloxane-treated titanium oxide
*14: Tetramethyl tetrahydrogen cyclotetrasiloxane-treated yellow iron oxide
*15: Tetramethyl tetrahydrogen cyclotetrasiloxane-treated red iron oxide
*16: Tetramethyl tetrahydrogen cyclotetrasiloxane-treated black iron oxide

In Table 1, the cosmetics of Examples 1 to 4, Example 6 and Comparative Examples 1 to 3 using polyoxyalkylene-modified silicone as component (A) are nanodisk emulsions, and the cosmetic of Example 5 using a polyoxyethylene hydrogenated castor oil derivative as component (A) is a vesicle emulsion.

As indicated in the table, the cosmetics of Examples 1 to 6, which contained pigments surface-treated with treatment agents containing amino acids, all had excellent vibration stability and cosmetic effects.

Meanwhile, the cosmetics of Comparative Examples 1 to 3, which contained pigments surface-treated with treatment agents not containing amino acids, were not able to achieve sufficient vibration stability, even when changing the types of UV absorbing agents that were blended.

### Formulation Examples

Hereinafter, formulation examples of the oil-in-water emulsion cosmetic of the present invention will be described. Needless to say, the present invention is not limited, in any way, by these formulation examples, and is defined by the claims. The blended amounts are all indicated in percentage by mass relative to the overall amount of the product.

Formulation Example 1. Foundation

| | Blended |
|---|---|
| | amount |
| Component name | (% by mass) |
| Water | balance |
| Trisodium EDTA | 0.05 |
| Glycerin | 2 |
| 1,3-Butylene glycol | 8 |
| Phenoxyethanol | 0.5 |
| (Dimethylacrylamide/sodium acryloyldimethyl taurate) | 0.5 |
| crosspolymer | |
| Stearoxy hydroxypropyl methylcellulose | 0.1 |
| Xanthan gum | 0.1 |
| Sodium stearoylmethyl taurate | 0.01 |
| Ethanol | 5 |
| PEG-12 dimethicone | 1.5 |
| Diisopropyl sebacate | 8 |
| Amino acid/ester (isostearyl sebacate, disodium stearoyl | |
| glutamate, aluminum hydroxide)-treated titanium oxide | 5 |
| Amino acid/ester (isostearyl sebacate, disodium stearoyl | |
| glutamate, aluminum hydroxide)-treated yellow iron oxide | 1 |
| Amino acid/ester (isostearyl sebacate, disodium stearoyl | |
| glutamate, aluminum hydroxide)-treated red iron oxide | 4 |
| Amino acid/ester (isostearyl sebacate, disodium stearoyl | |
| glutamate, aluminum hydroxide)-treated black iron oxide | 0.1 |
| Ethylhexyl salicylate | 5 |
| Octocrylene | 5 |
| Homosalate | 5 |
| Sorbitan sesquiisostearate | 0.5 |
| Isostearic acid | 0.5 |
| Silica/silicone-treated zinc oxide | 12 |

Formulation Example 2. Foundation

| | Blended |
|---|---|
| | amount |
| Component name | (% by mass) |
| Water | balance |
| Trisodium EDTA | 0.05 |
| Glycerin | 2 |
| 1,3-Butylene glycol | 8 |
| Phenoxyethanol | 0.5 |
| (Dimethylacrylamide/sodium acryloyldimethyl taurate) | 0.5 |
| crosspolymer | |
| Stearoxy hydroxypropyl methylcellulose | 0.1 |
| Xanthan gum | 0.1 |
| Sodium stearoylmethyl taurate | 0.01 |
| Ethanol | 5 |
| PEG-12 dimethicone | 1.5 |
| Diisopropyl sebacate | 8 |
| Amino acid/ester (isostearyl sebacate, disodium stearoyl | |
| glutamate, aluminum hydroxide)-treated titanium oxide | 5 |
| Amino acid/ester (isostearyl sebacate, disodium stearoyl | |
| glutamate, aluminum hydroxide)-treated yellow iron oxide | 1 |
| Amino acid/ester (isostearyl sebacate, disodium stearoyl | |
| glutamate, aluminum hydroxide)-treated red iron oxide | 4 |
| Amino acid/ester (isostearyl sebacate, disodium stearoyl | |
| glutamate, aluminum hydroxide)-treated black iron oxide | 0.1 |
| Ethylhexyl salicylate | 5 |
| Octocrylene | 5 |
| Homosalate | 5 |
| t-Butyl methoxy dibenzoylmethane | 2 |
| Sorbitan sesquiisostearate | 0.5 |
| Isostearic acid | 0.5 |

1. An oil-in-water emulsion cosmetic containing:
(A) a vesicle-forming amphiphilic substance;
(B) an oil component containing a UV absorbing agent other than ethylhexyl methoxycinnamate; and
(C) an amino acid surface-treated pigment.

2. The oil-in-water emulsion cosmetic according to claim 1, wherein the UV absorbing agent contains one or more types selected from among salicylic acid derivatives and β,β-diphenyl acrylate derivatives.

3. The oil-in-water emulsion cosmetic according to claim 1, substantially not containing ethylhexyl methoxycinnamate.

4. The oil-in-water emulsion cosmetic according to claim 1, wherein:
when the component (A) is polyoxyalkylene-modified silicone, the oil-in-water emulsion cosmetic further contains
(D) an aqueous component selected from among monohydric alcohols and dihydric alcohols, and
(E) an ionic surfactant;
the (D) aqueous component, when a monohydric alcohol is used, constitutes 1% to 15% by mass relative to the overall amount of the cosmetic, when a dihydric alcohol is used, constitutes 1% to 20% by mass relative to the overall amount of the cosmetic, and when a combination of a monohydric alcohol and a dihydric alcohol is used, the total amount thereof constitutes 1% to 45% by mass relative to the overall amount of the cosmetic; and
the (E) ionic surfactant is an anionic surfactant.

5. The oil-in-water emulsion cosmetic according to claim 1, containing an (F) whitener.
